(19) United States
(12) Patent Application Publication
(10) Pub. No.: US 2022/0127559 A1
(43) Pub. Date: Apr. 28, 2022

(54) Microrobot and Method of Manufacturing the Microrobot

(71) Applicant: Daegu Gyeongbuk Institute of Science and Technology, Daegu (KR)

(72) Inventors: Hong Soo Choi, Daegu (KR); Seung Min NOH, Gwangju (KR); Jin Yuong KIM, Daegu (KR); SungWong JEON, Daejeon (KR)

(73) Assignee: Daegu Gyeongbuk Institute of Science and Technology, Daegu (KR)

(21) Appl. No.: 17/500,392

(22) Filed: Oct. 13, 2021

(30) Foreign Application Priority Data
Oct. 27, 2020 (KR) 10-2020-0139899

(51) Int. Cl. C12N 1/04; A61K 47/69

(52) U.S. Cl. CPC C12N 1/04; B82Y 5/00; A61K 47/6929

(57) ABSTRACT

A microrobot is formed by mixing a biodegradable first material, biocompatible magnetic nanoparticles, and a drug, and includes a structure body having a three-dimensional (3D) structure and cells cultured on the surface of the structure body three-dimensionally.

### CROSS-REFERENCE TO RELATED APPLICATION

This application claims the benefit of Korean Patent Application No. 10-2020-0139899 filed on Oct. 27, 2020, and in the Korean Intellectual Property Office, the entire disclosure of which is incorporated herein by reference for all purposes.

### BACKGROUND

### 1. Field of the Invention

One or more example embodiments relate to a microrobot and a method of manufacturing the microrobot.

### 2. Description of the Related Art

A drug delivery system (DDS) is a system designed to efficiently deliver a required amount of a drug to minimize side effects and maximize the efficacy and effectiveness of the drug. Methods of delivering drugs may be classified by delivery route, drug type, and delivery technology form. The methods include, for example, an injection and infusion method developed in the 1960s, a suppository method developed in the 1970s, a nasal and oral administration method developed in the 1980s, and a direct delivery method of delivering a drug directly to the skin, lungs, and mouth developed in the 1990s. The DDS may reduce the time and cost needed to develop a new drug and be highly likely to be successful, and has thus become one of the advanced technologies into which developed countries started conducting active research from the 1970s. Korea started the research in earnest back in the 1990s.

The DDS may include a sustained release DDS (SRDDS), a controlled release DDS (CRDDS), and a targeted DDS (TDDS). The SRDDS may use a formulation designed to reduce a drug release rate to prevent low bioavailability or prevent extremely slow drug absorption or extremely fast drug excretion. The CRDDS may be designed to control an actual therapeutic effect by controlling a concentration of a target site which is mainly plasma, and may extend a drug delivery time and reproduce and predict a drug release rate as the SRDDS does. The TDDS may inhibit a non-specific distribution such that a drug is selectively delivered only to a cancer cell when a chemotherapeutic agent is used because the agent may be highly toxic even to a normal cell, thereby protecting a non-target site and delivering a drug only to a target site.

The TDDS may deliver a drug to a target site using nanorobots or microrobots. Currently, fundamental research is being actively conducted on the use of micro/nanotechnology-based micro/nanorobotics for medical purposes. For example, in the case of anticancer drugs, a large-scale market is expected to be formed, and cancer treatment methods using existing anticancer drugs have some issues such as non-selective toxicity and related side effects due to low targeted performance. Thus, there is active research on such a micro/nanotechnology-based DDS that may minimize side effects and maximize the efficacy of existing drugs and on the commercialization of the DDS. In addition, over the last few decades, biocompatible polymer-based nanoparticles have been developed as a representative targeted drug delivery platform, and various studies have been conducted to improve drug solubility, improve drug content while preventing drug loss, increase target delivery ability, and enhance drug release properties at an active site.

Recently, active research is being conducted to improve in-vitro or in-vivo drug delivery performance and effects of anticancer drugs by developing various types of intelligent nanoparticle formulations that release a drug in response to various environments (e.g., pH, oxidation-reduction reaction, temperature, magnetic field, light, etc.) of a living body.

The above description is information the inventor(s) acquired during the course of conceiving the present disclosure, or already possessed at the time, and is not necessarily art publicly known before the present application was filed.

### SUMMARY

Example embodiments provide a microrobot and a method of manufacturing the microrobot that effectively deliver a drug.

Additional aspects of example embodiments are not limited to what is described in the foregoing, and other aspects that are not described above may also be learned by those skilled in the art from the following description.

A microrobot and a method of manufacturing the microrobot will be described according to an example embodiment.

According to an aspect, there is provided a microrobot a structure body having a three-dimensional (3D) structure formed by mixing a biodegradable first material, biocompatible magnetic nanoparticles, and a drug, and cells cultured on a surface of the structure body three-dimensionally.

The structure body may be formed in a porous structure. The first material may include a biodegradable and photocurable material. For example, the first material may include gelatin methacryloyl (GelMA).

The structure body may further include a photoinitiator.

According to another aspect, there is provided a method of manufacturing a microrobot, including forming a mixture by mixing a biodegradable first material, biocompatible magnetic nanoparticles, and a drug, forming a structure body having a 3D structure by performing ultraviolet (UV) irradiation on the mixture, and culturing cells on a surface of the structure body three-dimensionally by culturing the structure body and the cells.

The culturing of the cells may include culturing the structure body and the cells simultaneously in a U-bottom well treated with an ultra-low attachment (ULA) surface for preventing cell adhesion.

The forming of the structure body may include forming the structure body using any one of microfluidic channel-based manufacturing, two-photon laser lithography, and 3D printing.

The structure body may be formed in a 3D porous structure.

The first material may include a biodegradable and photocurable material. The biodegradable and photocurable material may include GelMA.

According to example embodiments, a microrobot of which a 3D position is controllable through a magnetic field applied from outside may move wirelessly to a target position inside the body, thereby enabling a safe and precise movement inside the body of a patient.

In addition, the microrobot may store therein a drug and enable drug treatment to act only on a target.

Further, the microrobot may attach cells to a surface of a structure body to enable targeted treatment, and deliver a sufficient number of cells to a target to improve the efficiency of the treatment.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1A is a diagram illustrating a structure body of a microrobot according to an example embodiment;

FIG. 1B is a perspective view illustrating a microrobot according to an example embodiment; and

FIG. 2 is a diagram illustrating a method of manufacturing a microrobot according to an example embodiment.

### DETAILED DESCRIPTION

Hereinafter, example embodiments will be described in detail with reference to the accompanying drawings. It should be understood, however, that there is no intent to limit this disclosure to the particular example embodiments disclosed. On the contrary, example embodiments are to cover all modifications, equivalents, and alternatives falling within the scope of the example embodiments.

The terminology used herein is for the purpose of describing particular example embodiments only and is not to be limiting of the example embodiments. As used herein, the singular forms “a,” “an,” and “the,” are intended to include the plural forms as well, unless the context clearly indicates otherwise. As used herein, the term “and/or” includes any one and any combination of any two or more of the associated listed items. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

When describing the examples with reference to the accompanying drawings, like reference numerals refer to like constituent elements and a repeated description related thereto will be omitted. In the description of examples, detailed description of well-known related structures or functions will be omitted when it is deemed that such description will cause ambiguous interpretation of the present disclosure.

In addition, terms such as first, second, A, B, (a), (b), and the like may be used herein to describe components. Each of these terminologies is not used to define an essence, order, or sequence of a corresponding component but used merely to distinguish the corresponding component from other component(s). When it is mentioned that one component is “connected” or “accessed” to another component, it may be understood that the one component is directly connected or accessed to another component or that still other component is interposed between the two components. It should be noted that if it is described in the specification that one component is “directly connected” or “directly joined” to another component, still other component may not be present therebetween.

Example embodiments will be described in detail with reference to the accompanying drawings. When describing the example embodiments with reference to the accompanying drawings, like reference numerals refer to like components and a repeated description related thereto will be omitted.

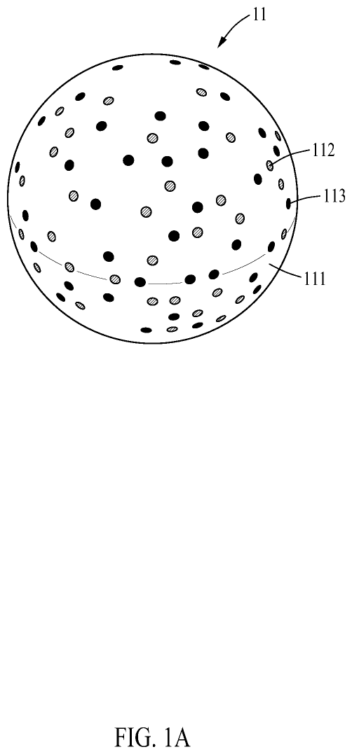

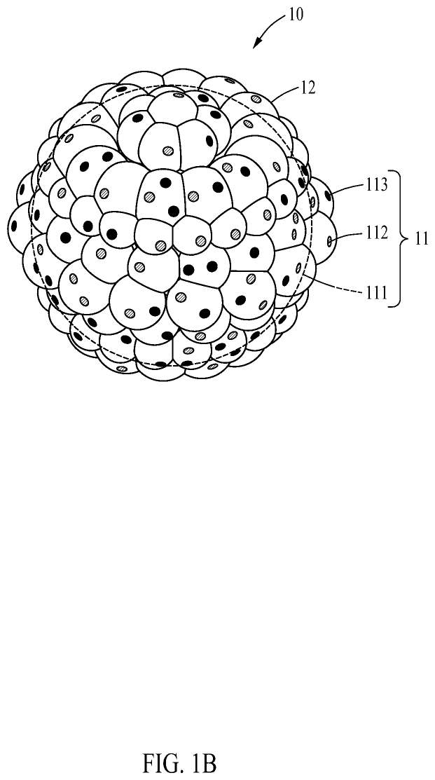

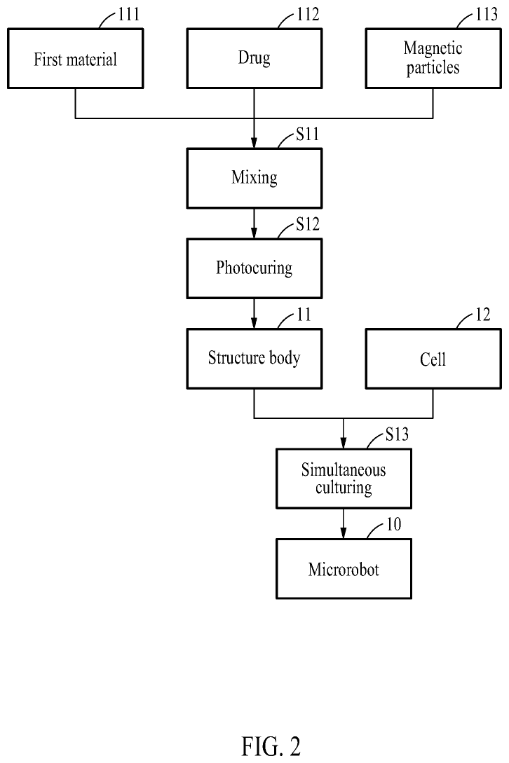

Hereinafter, a microrobot 10 and a method of manufacturing the microrobot 10 will be described with reference to FIGS. 1A and 1B, and 2. FIG. 1A is a diagram illustrating a structure body of the microrobot 10, and FIG. 1B is a perspective view illustrating the microrobot 10. FIG. 2 is a diagram illustrating a method of manufacturing the microrobot 10.

A microrobot used herein may refer to a micro-sized robot used for medical purposes, but is not limited thereto. The microrobot may also be a robot of nano or smaller size.

The microrobot 10 may include a micro- or nano-sized structure body 11 formed by mixing a first material 111 that is photocurable and biodegradable, a drug 112, and biocompatible magnetic nanoparticles 113, and cells 12 cultured on a surface of the structure body 11.

The structure body 11 may be formed using a three-dimensional (3D) printing method, a microfluidic channel-based manufacturing method, or a lithography method. In addition to the foregoing methods, other various methods may be used to form the structure body 11.

For example, using the 3D printing method, the structure body 11 and the microrobot 10 having a complex shape or high resolution may be formed. Using the microfluidic channel-based manufacturing method, for example, a droplet generation method, a plurality of spherical structure bodies 11 may be formed in a short period of time, and adjusting the size of the structure body 11 and the microrobot 10 may be facilitated. Using the lithography method that uses, for example, a two-photon laser, the structure body 11 and the microrobot 10 having high resolution and an ultrafine size may be formed.

The first material 111 may include gelatin methacryloyl (GelMA) that is photocurable and biodegradable.

GelMA may be photocurable and become biodegradable after being photocured. Thus, when the microrobot 10 formed with GelMA is administered into the body, it may less affect surrounding tissues or cells around a target and be decomposed in the body without a need to be retrieved afterward.

The drug 112 may be a drug or medicine to act on the target, and be selected according to a type of the target.

The magnetic nanoparticles 113 may be biocompatible, and use Fe3O4 particles, for example. The magnetic nanoparticles 113 may move the microrobot 10 to the target by a magnetic field applied from outside.

The cells 12 may be cultured on the surface of the structure body 11. For example, the cells 12 may be cultured along with the structure body 11 simultaneously in a U-bottom well treated with an ultra-low attachment (ULA) surface. Thus, the cells 12 may be attached to the surface of the structure body 11 in a 3D shape.

FIG. 2 is a diagram illustrating a method of manufacturing the microrobot 10.

In step S11, a mixture may be formed by mixing the first material 111 that is photocurable and biodegradable, the drug 112, and the magnetic nanoparticles 113. In step S12, ultraviolet (UV) irradiation may be performed on the mixture to form the structure body 11 having a 3D porous structure.

The structure body 11 may be formed using a 3D printing method, a microfluidic channel-based manufacturing method, or a lithography method. Other various methods may also be used to form the structure body 11 of micro or nano size.

The first material 111 may include gelatin methacryloyl (GelMA) that is photocurable and biodegradable.

In step S13, the structure body 11 and the cells 12 may be simultaneously cultured in a U-bottom well treated with an ULA surface such that the cells 12 are three-dimensionally cultured on the surface of the structure body 11 to grow thereon.

That is, the structure body 11 and the cells 12 may be simultaneously cultured in the U-bottom well with the ULA surface to allow the cells 12 to be attached onto the surface of the structure body 11 in a 3D form.

According to an example embodiment, culturing the cells 12 on the surface of the structure body 11 may provide a sufficient number of stem cells. In addition, the structure body 11 and the cells 12 may be stably attached to each other, and thus a loss of the cells 12 during migration in the body may be prevented. Thus, when reaching a target, the sufficient number of cells 12 may also be reached, which increases the effect and safety of the treatment.

According to example embodiments, the microrobot 10 may include the magnetic nanoparticles 113 that allows a 3D position control through a magnetic field applied from outside, and may thus wirelessly move into a position of a target T in the body. Thus, the microrobot 10 may move in the body of a patient safely and precisely. In addition, the microrobot 10 may be formed without using conventionally used metallic materials, for example, nickel and titanium, and thus be decomposed in the body with no need to be retrieved after the use.

The microrobot 10 may store therein a drug, and thus drug treatment may act only on a target. In addition, the microrobot 10 may have the cells 12 attached to the surface of the structure body 11, rather than being fixed between the structure bodies 11, thereby enabling targeted treatment. Further, the microrobot 10 may deliver a sufficient number of cells 12 to the target, and thus improve the efficiency of the treatment.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A microrobot, comprising:
a structure body having a three-dimensional (3D) structure formed by mixing a biodegradable first material, biocompatible magnetic nanoparticles, and a drug; and
cells cultured on a surface of the structure body three-dimensionally.

2. The microrobot of claim 1, wherein the structure body is formed in a porous structure.

3. The microrobot of claim 1, wherein the first material comprises a biodegradable and photocurable material.

4. The microrobot of claim 3, wherein the first material comprises gelatin methacryloyl (GelMA).

5. The microrobot of claim 1, wherein the structure body further comprises a photoinitiator.

6. A method of manufacturing a microrobot, comprising:
forming a mixture by mixing a biodegradable first material, biocompatible magnetic nanoparticles, and a drug;
forming a structure body having a three-dimensional (3D) structure by performing ultraviolet (UV) irradiation on the mixture; and
culturing cells on a surface of the structure body three-dimensionally by culturing the structure body and the cells.

7. The method of claim 6, wherein the culturing of the cells comprises:
culturing the structure body and the cells simultaneously in a U-bottom well treated with an ultra-low attachment (ULA) surface for preventing cell adhesion.

8. The method of claim 6, wherein the forming of the structure body comprises:
forming the structure body using any one of microfluidic channel-based manufacturing, two-photon laser lithography, and 3D printing.

9. The method of claim 6, wherein the structure body is formed in a 3D porous structure.

10. The method of claim 6, wherein the first material comprises a biodegradable and photocurable material,
wherein the biodegradable and photocurable material comprises gelatin methacryloyl (GelMA).
